(19) United States
(12) Patent Application Publication
(10) Pub. No.: US 2021/0278358 A1
(43) Pub. Date: Sep. 9, 2021

(54) Methods for Detecting Analytes in Excreta in an Analytical Toilet

(71) Applicant: Medic, Inc., Provo, UT (US)

(72) Inventors: David R. Hall, Provo, UT (US); K. Jeffrey Campbell, Spanish Fork, UT (US); Anthony E. Pullen, Tucson, AZ (US); Monte Johnson, Provo, UT (US)

(21) Appl. No.: 17/195,582

(22) Filed: Mar. 8, 2021

Related U.S. Application Data
(60) Provisional application No. 62/986,652, filed on Mar. 7, 2020.

(51) Int. Cl. G01N 27/12; G01N 33/543; G01N 33/569; B01L 3/00; E03D 9/00; G16H 10/40; G16H 50/70; G16H 15/00; G16H 40/67

(52) U.S. Cl. CPC G01N 27/128; G01N 33/54373; G01N 33/56983; B01L 3/502715; E03D 9/00; G16H 10/40; B01L 2200/141; G16H 15/00; G16H 40/67; G01N 2333/165; B01L 2200/027; B01L 2300/023; G16H 50/70

(57) ABSTRACT

Described is a method to detect an analyte in an analytical toilet that includes the steps of receiving excreta in a bowl, transporting a measured sample of the excreta through a passage and bringing the sample into contact with a sensor. The sensor comprises a FET configured to interact with an analyte in the excreta. When the sample is brought into contact with the sensor, the sensor indicates the presence of the analyte by a distinct electric signal.

### CROSS-REFERENCE TO RELATED APPLICATIONS

This application claims priority to U.S. Patent Application No. 62/986,652 titled “Methods for Testing Analytes in Excreta in an Analytical Toilet” filed on 7 Mar. 2020, which is incorporated herein by reference in its entirety.

### TECHNICAL FIELD

The present disclosure relates to analytical toilets. More particularly, it relates to analytical toilets equipped to provide health and wellness information related to excreta deposited by a user.

### BACKGROUND

The ability to track an individual's health and wellness is currently limited due to the lack of available data related to personal health. Many diagnostic tools are based on examination and testing of excreta, but the high cost of frequent doctor's visits and/or scans make these options available only on a very limited and infrequent basis. Thus, they are not widely available to people interested in tracking their own personal wellbeing.

Toilets present a fertile environment for locating a variety of useful sensors to detect, analyze, and track trends for multiple health conditions. Locating sensors in such a location allows for passive observation and tracking on a regular basis of daily visits without the necessity of visiting a medical clinic for collection of samples and data. Monitoring trends over time of health conditions supports continual wellness monitoring and maintenance rather than waiting for symptoms to appear and become severe enough to motivate a person to seek care. At that point, preventative care may be eliminated as an option leaving only more intrusive and potentially less effective curative treatments. An ounce of prevention is worth a pound of cure.

Just a few examples of smart toilets and other bathroom devices can be seen in the following U.S. Patents and Published Applications: U.S. Pat. No. 9,867,513, entitled “Medical Toilet With User Authentication”; U.S. Pat. No. 10,123,784, entitled “In Situ Specimen Collection Receptacle in A Toilet And Being in Communication With A Spectral Analyzer”; U.S. Pat. No. 10,273,674, entitled “Toilet Bowl For Separating Fecal Matter And Urine For Collection And Analysis”; US 2016/0000378, entitled “Human Health Property Monitoring System”; US 2018/0020984, entitled “Method Of Monitoring Health While Using A Toilet”; US 2018/0055488, entitled “Toilet Volatile Organic Compound Analysis System For Urine”; US 2018/0078191, entitled “Medical Toilet For Collecting And Analyzing Multiple Metrics”; US 2018/0140284, entitled “Medical Toilet With User Customized Health Metric Validation System”; US 2018/0165417, entitled “Bathroom Telemedicine Station.” The disclosures of all these patents and applications are incorporated by reference in their entireties.

One particular variety of detection, analysis, and trend tracking is related to biomarkers. “A bio-marker, or biological marker, is a measurable indicator of some biological state or condition. Biomarkers are often measured and evaluated to examine normal biological processes, pathogenic processes, or pharmacologic responses to a therapeutic intervention. Biomarkers are used in many scientific fields.” (See https://en.wikipedia.org/wiki/Biomarker). Biomarker information can be a valuable resource in providing for the health and wellness of an individual or population. Some of the uses include being used to detect a disease at its earliest stages and monitoring the progression of key health metrics over time.

Initially, the detection of biomarkers was performed on macro-scale samples, but as technology has improved, equipment both takes up a smaller footprint and is able to use smaller and smaller sample sizes. Additionally, as technology improved, smaller and smaller concentrations of individual biomarkers became detectable. Recent innovation has allowed for the creation of circuitry capable of detecting a variety of desirable, specific, individual molecular elements. Current testing of many biomarkers requires the sample to be processed in a laboratory or clinic. Such testing is also costly, inconvenient, and time consuming.

One such example is described in U.S. Pat. No. 7,301,199 “Nanoscale Wires and Related Devices”, which outlines the production of nanometer scale circuitry elements. This technology can be used to make nanoscale versions of numerous components. The '199 patent states “for example, semiconductor materials can be doped to form n-type and p-type semiconductor regions for making a variety of devices such as field effect transistors, bipolar transistors, complementary inverters, tunnel diodes, light emitting diodes, sensors, and the like.” (Page 5, Abstract). The disclosure of the '199 patent is incorporated herein in its entirety.

Additionally, US 2018/0088079 “Nanoscale Wires with External Layers for Sensors and Other Applications” disclosed further details related to producing sensors that use nanoscale wires. For example it teaches “Certain aspects of the invention are generally directed to polymer coating on nanoscale wires that can be used to increase sensitivity to analytes” (Page 1, Abstract). The disclosure of US 2018/0088079 is incorporated herein in its entirety.

### SUMMARY

In a first aspect, the disclosure provides a method to detect an analyte in an analytical toilet that includes the steps of receiving excreta in a bowl, transporting a measured sample of the excreta through a passage and bringing the sample into contact with a sensor. The sensor comprises a FET configured to interact with an analyte in the excreta. When the sample is brought into contact with the sensor, the sensor indicates the presence of the analyte by a distinct electric signal.

In a second aspect, the disclosure provides an analytical toilet comprising a bowl to receive excreta and a FET-based sensor. The sensor includes a component functionalized to interact with an analyte such as a biomarker. The toilet delivers to the component a sample of or made from the excreta. The component creates an electronic signal further creating data indicating if the analyte is present in the sample.

In a third aspect, the disclosure provides additional information related to delivering the sample to the component, including continuous and segmented flow.

In a fourth aspect, the disclosure provides additional information related to cleaning and preparing the sensor.

In a fifth aspect, the disclosure provides additional information on how to process the data.

Further aspects and embodiments are provided in the drawings, detailed description and claims.

### BRIEF DESCRIPTION OF THE DRAWINGS

The following drawings are provided to illustrate certain embodiments described herein. The drawings are merely illustrative and are not intended to limit the scope of claimed inventions and are not intended to show every potential feature or embodiment of the claimed inventions. The drawings are not necessarily drawn to scale; in some instances, certain elements of the drawing may be enlarged with respect to other elements of the drawing for purposes of illustration.

FIG. 1 illustrates an analytical toilet with the lid closed, according to an embodiment of the disclosure.

FIG. 2 illustrates an analytical toilet with lid open, according to an embodiment of the disclosure.

FIG. 3 illustrates an analytical toilet with lid closed and a portion of the exterior shell removed, according to an embodiment of the disclosure.

FIG. 4 further illustrates the interior of the toilet of FIGS. 1-3, according to an embodiment of the disclosure.

FIG. 5 illustrates a modular analytical test device attached to a manifold, according to an embodiment of the disclosure.

FIG. 6 illustrates another embodiment of a modular analytical test device.

FIG. 7 illustrates another embodiment of a modular analytical test device.

FIG. 8 illustrates sample flow to a sensor in a fluidic system according to an embodiment of the disclosure.

FIG. 9 illustrates sample flow to a sensor in another fluidic system, according to an embodiment of the disclosure.

### DETAILED DESCRIPTION

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

### Definitions

The following terms and phrases have the meanings indicated below, unless otherwise provided herein. This disclosure may employ other terms and phrases not expressly defined herein. Such other terms and phrases shad have the meanings that they would possess within the context of this disclosure to those of ordinary skill in the art. In some instances, a term or phrase may be defined in the singular or plural. In such instances, it is understood that any term in the singular may include plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure and are not meant to be limiting in any fashion. Nor do these phrases indicate any kind of preference for the disclosed embodiment.

As used herein, “toilet” is meant to refer to any device or system for receiving human excreta, including urinals.

As used herein, “bowl” refers to the portion of a toilet that is designed to receive excreta.

As used herein, the term “base” refers to the portion of the toilet below and around the bowl supporting it.

As used herein, the term “user” refers to any individual who interacts with the toilet and deposits excreta therein.

As used herein, the term “excreta” refers to any substance released from the body including urine, feces, menstrual discharge, and anything contained or excreted therewith.

As used herein, the term “manifold” is intended to have a relatively broad meaning, referring to a device with multiple conduits and valves to controllably distribute fluids, namely water, liquid sample and air.

As used herein, the term “test chamber” is meant to refer broadly to any space adapted to receive a sample for testing, receive any other substances used in a test, and apparatus for conducting a test, including any flow channel for a fluid being tested or used for testing.

As used herein, the term “sensor” is meant to refer to any device for detecting and/or measuring a property of a person or substance regardless of how that property is detected or measured, including the absence of a target molecule or characteristic.

As used herein, the term “microfluidics” is meant to refer to the manipulation of fluids that are contained to small scale, typically sub-millimeter channels. The “micro” used with this term and others in describing this invention is not intended to set a maximum or a minimum size for the channels or volumes.

As used herein, the term “microfluidic chip” is meant to refer to is a set of channels, typically less than 1 mm², that are etched, machined, 3D printed, or molded into a microchip. The micro-channels are used to manipulate microfluidic flows into, within, and out of the microfluidic chip.

As used herein, the term “microfluidic chamber” is meant to refer to a test chamber adapted to receive microfluidic flows and/or a test chamber on a microfluidic chip.

As used herein, the term “lab-on-chip” is meant to refer to a device that integrates one or more laboratory functions or tests on a single integrated circuit. Lab-on a chip devices are a subset of microelectromechanical systems (MEMS) and are sometimes called “micro total analysis systems” (μTAS).

As used herein, the term “data connection” and similar terms are meant to refer to any wired or wireless means of transmitting analog or digital data and a data connection may refer to a connection within a toilet system or with devices outside the toilet.

As used herein, “biomarker” and “biological marker” are meant to refer to a measurable indicator of some biological state or condition, such as a normal biological processes, pathogenic processes, or pharmacologic responses to a therapeutic intervention. Some biomarkers are related to individual states or conditions. Other biomarkers are related to groups or classifications or states or conditions. For example, a biomarker may be symptomatic of a single disease or of a group of similar diseases that create the same biomarker.

As used herein, “analyte” is meant to refer to a substance whose chemical constituents are being identified and measured.

As used herein, the prefix “nano-” is meant to refer to something in size such that units are often converted to the nano-scale for ease before a value is provided. For example, the dimensions of a molecule may be given in nanometers rather than in meters.

As used herein, “miniaturized electronic system” is meant to refer to an electronic system that uses nanometer scale technology.

As used herein, “FET” is meant to refer to a field effect transistor, which is a device which uses an electric field to control the current flowing through a device. FETs are also known by the name “unipolar transistor”.

As used herein, “functionalize” and similar variants are meant to refer to a device, especially a nanometer scale device, the surface of which being configured to interact with a specific analyte, such as a specific biomarker.

As used herein, “genomic derived signal” is meant to refer to a molecule generated by the genome of a cell, bacteria, virus, or other nucleic acid carrier, such as DNA, RNA, microRNA, cell-free (circulating) nucleic acids, or those of various immunologically related cells.

As used herein, a “fluidic circuit” is meant to refer to the purposeful control of the flow of a fluid. Often, this is accomplished through physical structures that direct the fluid flow. Sometimes, a fluidic circuit does not include moving parts.

As used herein, a “fluidic chip” is meant to refer to a physical device that houses a fluidic circuit. Often, a fluidic chip facilitates the fluid connection of the fluidic circuit to a body of fluid.

As used herein, an analyte that “interacts” with a sensor is meant to refer to several ways a component (e.g., receptor) of a sensor can detect the analyte. “Interacting” may include reversible binding of an analyte to a component in a sensor. This may also be referred to as labile binding where the analyte is weakly bound to a component in the sensor and can be removed by a removal treatment such as a flushing or cleaning process. “Interacting” may include irreversible binding of an analyte to a component in a sensor where the binding is a one-time event and the component, of the sensor or the entire sensor must be replaced after each use. “Interacting” may include a non-binding event wherein the analyte is in the vicinity of the component of the sensor such that the magnetic, optical or electrical properties of the component are perturbed by the presence of an analyte. For example, this may be caused by negative or positive charges located on the surface of the analyte.

### Exemplary Embodiments

The present disclosure relates to analytical toilets with analytical tools (may also be referred to as a “smart” or a “health and wellness” toilet) which detects, analyzes, and/or tracks the trends of analytes, such as biomarkers, of a user who deposits excreta into the toilet. More specifically, the toilet receives excreta from a user, processes the excreta in preparation for analysis, and brings a sample of excreta (including processed excreta) into a testing area for detection by a FET-based sensor. For example, a “gate” of a FET comprised of source, drain, gate, and bulk circuit connections has been functionalized to interact with a specific analyte, such as a biomarker, on a molecular or atomic level. The circuitry component provides, amplifies, attenuates, or otherwise modulates a data signal depending on whether the specific analyte is present in the excreta sample in contact with sensor. After the toilet has finished with the excreta, the toilet purges the excreta from the toilet in preparation for receiving a new excreta sample.

The invention disclosed herein provides methods of detection or determination of species. One embodiment involves a method of detecting an analyte, involving contacting a nanoscopic wire with a sample, and determining a property associated with the nanoscopic wire where a change in the property, when the nanoscopic wire is contacted with the sample, indicates the presence and/or quantity of the analyte in the sample. In another embodiment, the method involves contacting an electrical conductor, or a nanoscopic wire, with a sample, and determining the presence and/or quantity of an analyte in the sample by measuring a change in a property of the conductor resultant from the contact, where less than ten molecules of the analyte contribute to the change in the property.

In another embodiment, a method of the invention includes contacting a nanoscopic wire with a sample suspected of containing an analyte and determining a change in a property of the nanoscopic wire. In another embodiment, the method involves contacting a nanoscopic wire with a sample having a volume of less than about 10 μL, and measuring a change in a property of the nanoscopic wire resultant from the contact.

In accordance with the present disclosure, an analytical toilet that includes an infrastructure for multiple health and wellness analysis tools is provided. This provides a platform for the development of new analytical tools by interested scientists and companies. Newly developed tests and diagnostic tools may be readily adapted for use in a system having a consistent tool interface.

The analytical toilet may provide a fluid processing manifold that collects and routes samples from the toilet bowl to various scientific test devices and waste handling portals throughout the device. The analytical toilet may provide multiple fluid sources via the manifold system. The manifold may be adapted to connect to a plurality of analytic test devices adapted to receive fluids from the manifold. The manifold may be designed to selectively provide a variety of different fluid flows to the analytical test device. These fluids may comprise, among others, excreta samples, buffer solutions, reagents, water, cleaners, biomarkers, dilution solutions, calibration solutions, and gases such as air or nitrogen. These fluids may be provided at different pressures and temperatures. The manifold and analytical test device may also be adapted to include a fluid drain from the analytical test devices.

The manifold system may provide a standardized interface for analytical test devices to connect and receive all common supplies (e.g., excreta samples, flush water), data, and power. Common supplies may be supplied from within (e.g., reagents, cleaners) or without (e.g., water) the toilet system. The analytical test devices may be designed to receive some or all of the standardized flows. The analytical test devices may also include storage cells for their own unique supplies (e.g., test, reagent).

The manifold may be adapted to direct fluids from one or more sources to one or more analytical test devices. The manifold and analytical test, devices may be designed such that analytical test devices can be attached to and detached from the manifold making them interchangeable based on the needs of the user. Different analytical test devices may be designed to utilize different test methods and to test excreta samples for different constituents.

The analytical toilet may provide an electrical power connection and a data connection for the analytical test device. The electrical power and data connections may use the same circuit. The toilet may be provided with pneumatic and/or hydraulic power to accommodate the analytical test devices. The toilet platform can perform various functions necessary to prepare samples for examination. These functions include, but are not limited to, diluting or concentrating samples, particle filtration, sample agitation, pH normalization, normalization based on the concentration of one or more analytes, normalization based on prior measurements taken by toilet apparatus, and adding reagents.

The analytical toilet may also provide, among other things, fluid transport, fluid metering, fluid valving, fluid mixing, separation, analyte amplification, fluid storage, fluid incubation and fluid release and disposal. The toilet may also be equipped to provide cleansers, sanitizers, rinsing, and flushing of all parts of the system to prevent cross-contamination of samples. The system may produce electrolyzed water for cleaning.

One layer of the fluidic manifold may be dedicated to macro-scale mixing of fluids. Sample, diluents, and reagents can be available as inputs to the mixers. The mixing chamber may be placed in series with all other scientific test devices, allowing bulk mixed sample to be routed to anywhere from one to all stations (i.e., analytical test device interfaces) for analysis. Mixing may also occur in an analytical test device.

The samples may be filtered for large particulates at the fluid ingress ports of the manifold. The fluid manifold may use a network of horizontal and vertical channels along with simple valves to route urine or prepared stool samples to one of several scientific test devices located on the platform.

The manifold may be constructed using additive layers, and different layers can be customized for particular applications. Standard ports and layouts may be used for interfacing with external components, such as pressure sources and flow sensors. In general, characteristic channel volumes at the bottom of the manifold stack may be on the order of milliliters. At the top of the manifold stack may be the microfluidic science device, which will interface simultaneously with multiple microfluidic chips using standardized layout and pressure seals.

The analytic test devices may be designed to perform one or more of a variety of laboratory tests. Any test that could be performed in a medical or laboratory setting may be implemented in an analytical test device. These tests may include measuring pulse, blood pressure, blood oxygenation, electrocardiography, body temperature, body weight, excreta content, excreta weight, excreta volume, excreta temperature, excreta density, excreta flow rate, and other health and wellness indicators.

The analytical toilet system may be adapted to work with a variety of actuation technologies that may be used in the analytical test devices. The system may provide electronic and fluidic interconnects for various actuator technologies and supports OEM equipment. The system may be adapted to work with actuator modules that can be attached to the sample delivery manifold and controlled by a central processor. The system platform may support an inlet and outlet for the pressure transducer that interfaces with the fluidic manifold, and electronic or pneumatic connections where required. The system may support a variety of macro- and microfluidic actuation technologies including, but not limited to, pneumatic driven, mechanical pumps (e.g., peristaltic), on-chip check-valve actuators (e.g., piezo-driven or magnetic), electroosmotic driven flow, vacuum pumps, and capillary or gravity driven flow (i.e., with open channels and vents).

One benefit of the present disclosure is the detection, monitoring, and tracking of a user's biomarkers without having any inconvenience aside from what they would otherwise do using the toilet. Without, the present, disclosure, among other things, people often have to manually collect samples of excreta, use equipment they are less familiar with than a toilet, or wait longer for analysis and results. Each of these things can negatively impact a user's experience and/or the quality or accuracy of the results.

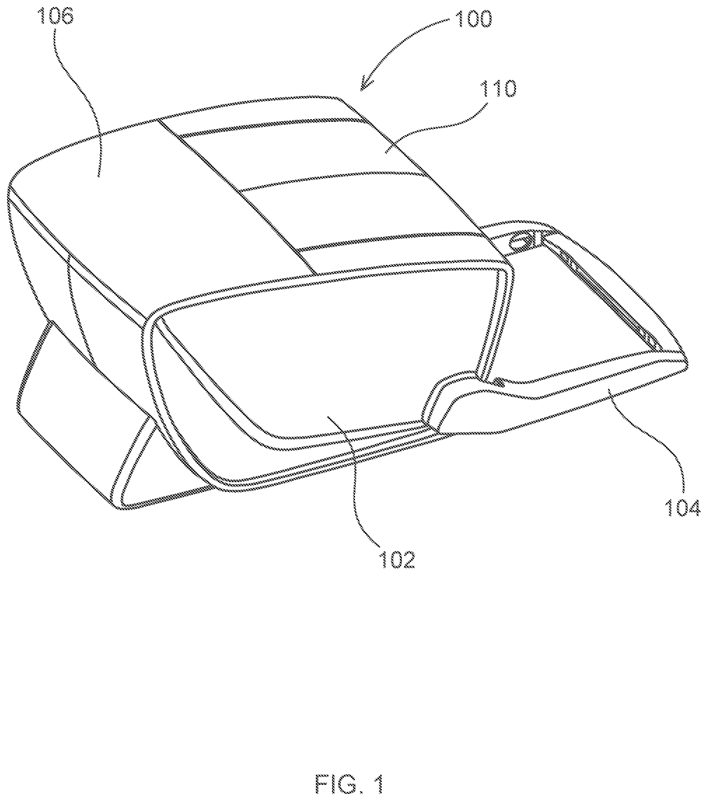

Now referring to FIGS. 1-3, a preferred embodiment of an analytical toilet 100 is shown. FIG. 1 illustrates the analytical toilet 100 with the lid 110 closed, according to an embodiment of the disclosure. FIG. 1 further shows exterior shell 102, foot platform 104 and rear cover 106. The lid 110 is closed to prevent a user from depositing excreta in toilet 100 until the toilet is ready for use.

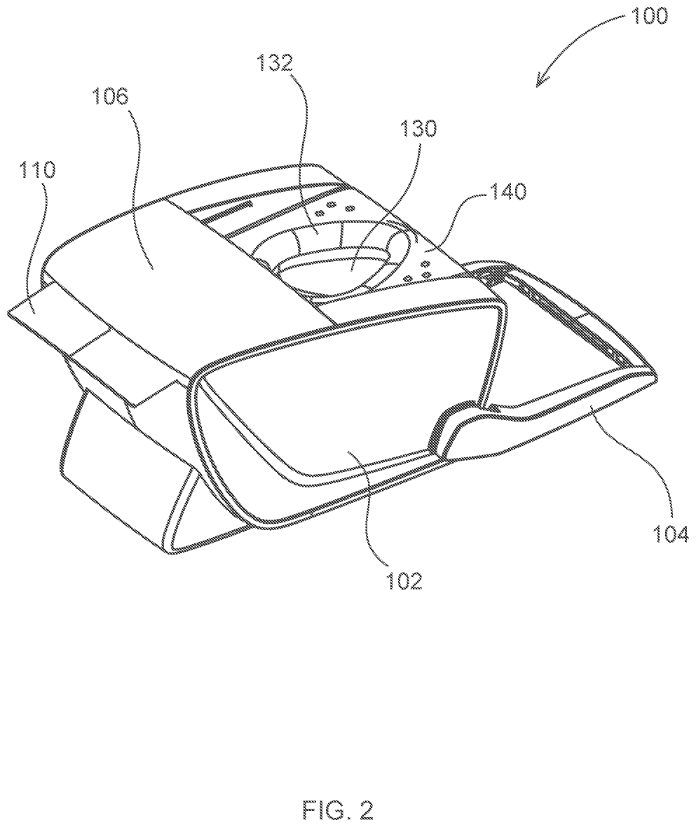

FIG. 2 illustrates toilet 100 with lid 110 open, according to an embodiment of the disclosure. Toilet 100 includes exterior shell 102, rear cover 106, bowl 130, seat 132, lid 110, fluid containers 140 and foot platform 104. Housed within toilet 100 are a variety of features, including equipment, that facilitate receiving excreta, processing excreta for analysis, analyzing excreta, and disposing of excreta. FIG. 2 shows toilet 100 with lid 110 open so a user can sit on seat 132 and deposit excreta in toilet 100.

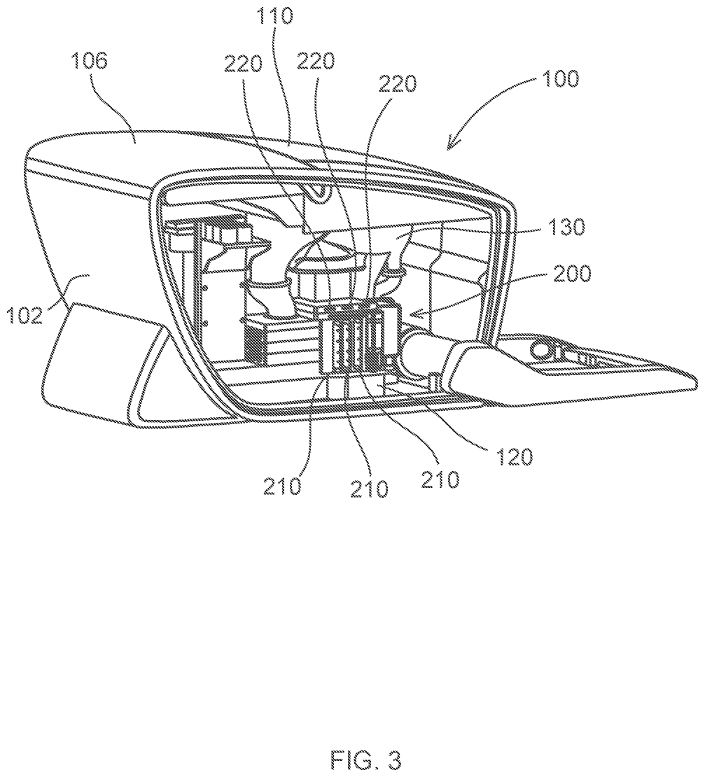

FIG. 3 illustrates toilet 100 with lid 110 closed and a portion of exterior shell 102 removed, according to an embodiment of the disclosure. This allows access to equipment housed within toilet 100. With exterior shell 102 removed, base 120 and manifold area 200 is visible. Manifold area 200 includes test areas 210 and fluidic chip slots 220. Preparation and/or analysis of sample can selectively take place in a test area 210 or fluidic chip slot 220. Manifold area 200 is the area where analysis takes place.

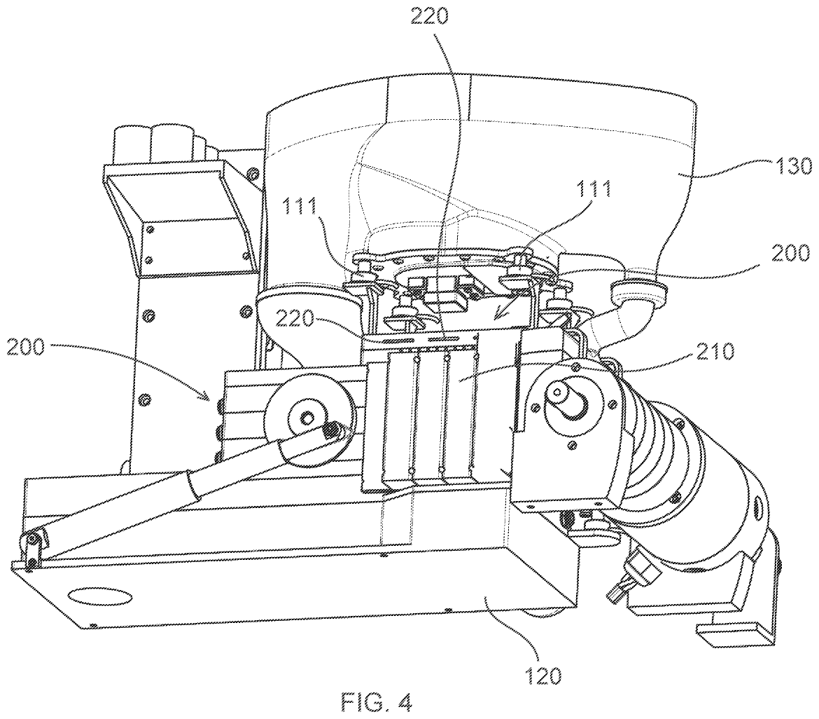

FIG. 4, further illustrates the interior of the toilet of FIGS. 1-3, according to an embodiment of the disclosure. The internal components of the toilet 100 are supported by a base 120. The bowl 130 is supported by one or more load cells 111. A manifold 200 is located below the bowl 130. The manifold 200 comprises a plurality of fluid paths. These fluid paths allow the manifold 200 to move fluids between the bowl 130, fluid containers 140, outside sources (e.g., municipal water supplies), other sources (e.g., air or water electrolyzing unit), analytical test devices 210, and the toilet outlet. The manifold 200 also provides electrical power and data connections to the analytical test devices 210. The manifold 200 can also directly pass fluids and/or solids from the bowl 130 to the toilet outlet.

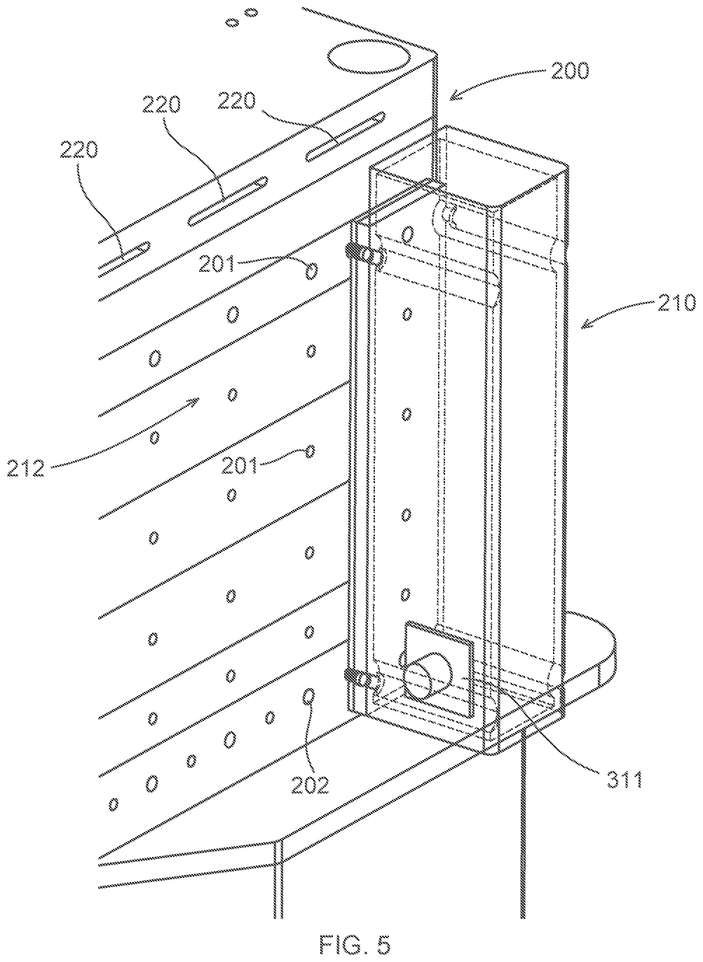

FIG. 5 illustrates a modular analytical test device 210 attached to a manifold 200, according to an embodiment of the disclosure. The manifold 200 is adapted to provide receptacles 210 with standardized connection interfaces for multiple analytical test devices 210. The manifold 200 is shown here with multiple fluid sources 201 for the analytical test device 210. In various embodiments, the manifold 200 may include receptacles 212 for more than one type of analytical test device 210 (e.g., different sizes, fluid supply needs, etc.). Slots 220 are also shown where microfluidic chips (MFCs) that further comprise sensor components may be inserted.

In various exemplary embodiment, the analytical test, device 210 includes multiple inlets in fluid communication with the manifold 200. The selected fluid flows are directed into a test chamber with one or more sensors 311 (flow channels internal to the analytical test device not shown in FIG. 5). The sensors 311 may be one or more of electrochemical sensors, spectrometers, chromatography, CCD (charge-coupled device), or metal oxide semiconductor field-effect transistor (MOSFET) including complementary metal oxide semiconductor field-effect transistor (CMOSFET). The analytic test device 210 also includes at least one outlet 202 or drain in fluid communication with the manifold 200.

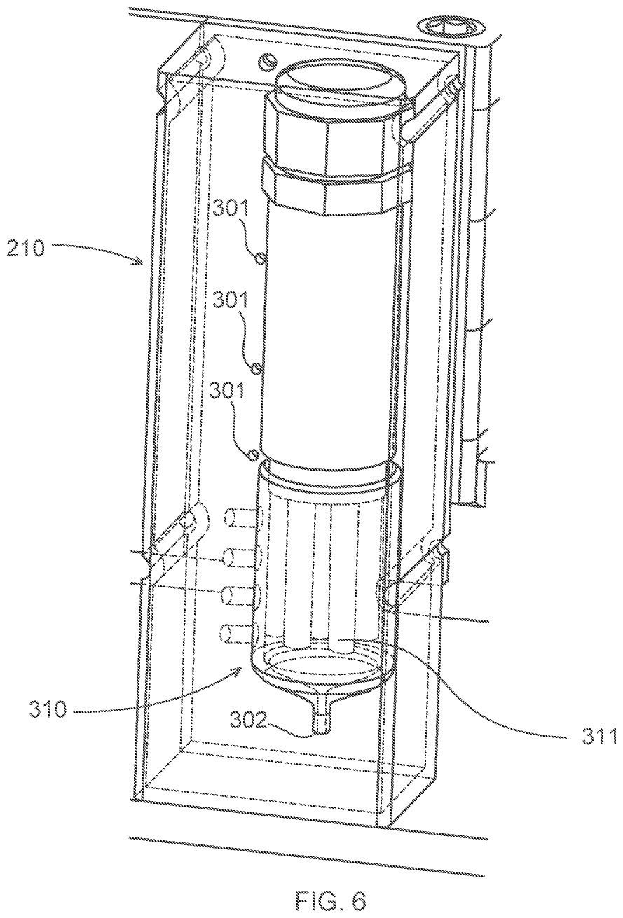

FIG. 6 illustrates another embodiment of a modular analytical test device 210. The analytical test device 210 includes multiple fluid inlets 301, test chamber 310, and at least one fluid outlet 302. The analytic test device 210 includes a test chamber 310 that receives fluid flows and contains at least one array of sensors 311.

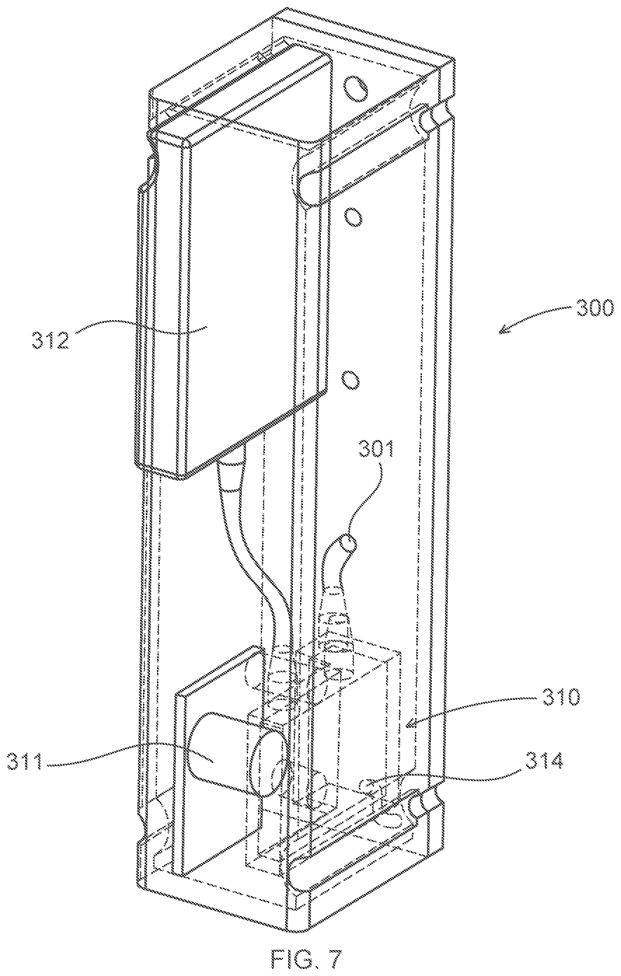

FIG. 7 illustrates another embodiment of a modular analytical test device 210. The analytical test device 210 includes multiple fluid inlets 301, test chamber 310, and at least one fluid outlet 314. This embodiment of an analytical test device 210 includes a storage cell 312, also in fluid communication with the test chamber 310. The analytical test device 210 may also include a pump to move fluid (e.g., test reagent) from the cell 312 to the test chamber 310. The analytic test device 210 also includes a camera adjacent to the test chamber 310 to monitor the contents of the test chamber 310. In various embodiments, the test chamber 310 is used to mix an excreta sample with a reagent that will cause a color change if a target analyte is present in the excreta sample. The camera is adapted to detect the color change, in various exemplary embodiments, the camera may be used to observe other characteristics or changes to the sample in the test chamber 310 (e.g., urine settling).

An analytical toilet, such as toilet embodiment 100, may further comprise a field effect transistor (FET) based sensor. There are many ways to incorporate the FET-based sensor into the toilet, the selection of which will depend on various factors, including ease of manufacture and maintenance, target market, physical constraints, frequency of use compared to other desired functions of the toilet, and cost. In one preferred embodiment, the FET-based sensor is built into a fluidic circuit. More preferably, the fluidic circuit is on a fluidic card. Still more preferably, the fluidic circuit on the fluidic card is a microfluidic circuit, on a micro fluidic card. Preferably, the fluidic card comprising the sensor is inserted into a slot or receptacle of the toilet which connects the fluid circuit on the card to the toilet's fluidic delivery system, enabling the card to receive the sample derived from the excreta. Alternatively, the sensor is part of a larger device that may be attached to the toilet, such as a device that processes and/or analyzes excreta. Alternatively, the FET-based sensor is built into the toilet rather than being on a card. Alternatively, the sensor is external to the remainder of the toilet and is connected to receive and/or return fluid from the toilet, such as may be accomplished by connecting the sensor to part of the toilet with tubes or pipes.

In various exemplary embodiments, microfluidic systems may be used to isolate and transport a sample, add and mix reagents if appropriate, and test the sample for one or more biomarkers on a small scale (i.e., sub-millimeter scale) in an analytical toilet described herein. The microfluidic system may comprise an open microfluidic system, continuous-flow microfluidic system, droplet-based microfluidic system, digital microfluidic system, nanofluidic system, paper-based microfluidic system, or combinations thereof.

A microfluidic FET-based analyte detection system may be located on a microfluidic chip (MFC). In a preferred embodiment, the MFC includes a test chamber with a lab-on-chip (“LoC”) (also known as “test-on-chip”). The LoC may be designed to perform one or more laboratory tests. In various exemplary embodiments, one or more microfluidic chips (MFCs) may be removed or added to the toilet system as desired or needed at any given time, such as for different biomarker tests. In an exemplary embodiment, a DNA microfluidic chip may be used as a component in a biomarker sensor in a health and wellness analytical toilet. The DNA chip may comprise a DNA microarray, such as the GenChip DNAarray (Affymetrix, Santa Clara, Calif., USA). The DNA microarray comprises one or more pieces of DNA (probes) for biomarker detection. The MFC may comprise one or more affixed proteins in an array-like fashion. In an exemplary embodiment, the proteins are monoclonal antibodies for detection of antigens.

In a preferred embodiment, a component of the FET-based sensor is functionalized to interact with an analyte, such as a biomarker, and produce a signal based on the presence and/or concentration of the biomarker. Often, this means the sensor is configured to respond to an individual molecule or even a specific molecular element or portion of a biomarker. The sensed analytes, such as biomarkers, may be indicative of cancer, infection, disease, drug overdose, drug impairment or injury. Biomarker analytes include immunological genomic derived signals, DNA genomic derived signals, RNA genomic derived signals, microRNA genomic derived signals, other genomic derived signals, proteins, carbohydrates, lipids, metabolites, and ionic concentrations. Other analytes that may be sensed are viruses (e.g., COVID-19), bacteria, alcohol, prescription drugs, illicit drugs and recreational drugs. In some preferred embodiments, the component of the sensor amplifies the concentration of the targeted analyte. In other preferred embodiments, the component dilutes the concentration of the targeted analyte. In some preferred embodiments, the concentration is neither amplified nor diluted. Use of one category of tests to detect a particular analyte does not preclude use of another test category to detect or measure the same analyte.

One exemplary class of sensors are biosensor field-effect transistors (BioFETs). BioFETs are based on metal-oxide-semiconductor field effect transistors (MOSFETs) that are gated by changes in the surface potential induced by the binding of biomolecules. Complimentary metal-oxide-semiconductor field effect transistors (CMOSFETs) may also be used. BioFETs comprise a field effect transistor and a biological recognition element or receptor.

BioFET-based sensors for a health and wellness analytical toilet may comprise one or more nanowires or functionalized nanowires to bind with a biomarker, one or more nanocrystals or functionalized nanocrystals, one or more sheets of graphene or functionalized graphene or a combination thereof. These materials are placed in a manner in the FET to bridge the source and drain electrodes. The BioFET may comprise a semiconductor with a functionalized gate. Other sensors include colorimetric based assays, paper-based analytical devices, a luminescent markers or labels, and a fluorescent or otherwise optically stimulated marker or label.

In some embodiments, nanowires for use in BioFETs may include conducting polymers such as polythiophene, polyaniline, polycarbazole, poly(3,4-ethylenedioxythiophene), polypyrrole, polyphenol or combinations thereof. Nanowires may comprise metals such as germanium, silver, gold, platinum, nickel palladium or combinations thereof. Nanowires may comprise two or more metals in a core-shell like arrangement. The metallic nanowires may comprise a thin oxide surface layer for covalent attachment of biomarker receptors. Nanowires may include inorganic oxide materials such as indium oxide (In₂O₃), indium tin oxide (ITO), zinc oxide (ZnO), tin oxide (SnO), titania (TiO₂) or silica (SiO₂). In an exemplary embodiment, the nanowire comprises a non-functionalized or functionalized single walled carbon nanotube (SWCNT) or a non-functionalized or functionalized multi-walled carbon nanotube (MWCNT) or a combination thereof. In a more exemplary embodiment, the nanowire comprises silicon (Si). The Si nanowire may comprise p-type or n-type Si. The Si nanowires may have a diameter of about 2 nm or larger. In other embodiments, the Si nanowires may have a diameter of about 2-100 nm. In an exemplary embodiment, the diameter of the Si nanowire may be in the range of about 2-30 nm. The nanowire used may have an aspect ratio of length to diameter in a range of about 500-1500. The nanocrystals may comprise colloidal metal, such as gold, or quantum dots. The nanocrystals may comprise semiconducting or super paramagnetic metal oxides such as iron oxides. Some variations include multiple sensors per component that detect the same biomarker, diverse concentration strengths of the same biomarker, and combinations of multiple biomarkers in an array or assay panel.

The conducting polymers, nanowires and nanocrystals used in FET-based sensors for use in a health and wellness analytical toilet described herein may be exploited for their optical, magnetic and electrical properties to detect various analytes. Their optical, magnetic and electrical properties may be tuned based on their size, how they are made, composition and how they are functionalized. A variety of transduction methods may be used to convert a binding event of a biomarker to a component in a sensor to a detectable and monitorable digital signal. The digital signal may comprise conductivity, resistance, voltage, conductance, fluorescence, spectroscopic, pH, magnetic changes or a combination thereof. In an exemplary embodiment, conductance or voltage or both the conductance and voltage in a FET-based sensor may be monitored when sensing for a biomarker. The conductance or voltage or both the conductance and voltage may be monitored with respect to time when a biomarker interacts with the sensor.

A component of a FET-based sensor, such as a nanowire or conducting polymer, may be functionalized with one or more monoclonal antibody receptors. The receptors may be covalently attached. Antibody receptors may be used to detect one or more viruses. Such viruses may include DNA, RNA or reverse transcribing viruses. An individual sensor may comprise only one type of antibody to target and detect a specific virus, such as influenza A, adenovirus, COVID-19 or Ebola. In other embodiments, a sensor may comprise two or more antibodies to target and detect two or more different types of viruses.

A component of a FET-based sensor, such as a nanowire or conducting polymer, may be functionalized with one or more monoclonal antibodies to detect pathogens that cause diseases such as cancer. Cancerous tumor cells release antigens that can be detected. These antigens may be proteins, peptides or polysaccharides. In an exemplary embodiment, a FET-based sensor in an analytical toilet may comprise one or more antibodies to detect antigens released by cancerous cells. Antigens are biomarkers released by cancerous cells may also be referred to as tumor markers. Such biomarkers may include CA 15-3 from breast cancer cells. Prostate specific antigen (PSA) found in prostate cancer cells. CA-125 antigen biomarker commonly found in ovarian cancer cells. Carcinoembryonic antigen (CEA) found in colorectal cancer cells.

A component in a FET-based sensor may be functionalized with peptide nucleic acid (PNA). PNA can be used as a gene sensor. A PNA is a non-charged variant of DNA and has high selectivity toward complementary DNA sequences. A PNA sensor is very sensitive with almost no electrochemical response toward DNA with one base mismatch. A PNA-based sensor may be used for detection of the DNA sequence responsible for sickle cell anemia.

A FET-based sensor in an analytical toilet described herein may be able to detect one or more viruses. The detectable viruses may be from the coronavirus class including alphacoronoavirus, betacoronovirus, gammacoronavirus or deltacoronavirus. More specifically, these viruses may include SARS-CoV-2 (also known as COVID-19) or SARS-CoV. A component in a FET-based sensor may be functionalized with angiotensin converting enzyme 2 (ACE2) antibody as a receptor. The ACE2 receptor interacts and binds with the spike proteins on the surface of the SARS-COV-2 virus.

FET-based sensors used in a health and wellness analytical toilet described herein are capable of multiplexed detection. Multiplexed detection is necessary for simultaneous detection of multiple biomarkers such as proteins. This is critical for reliable detection of complex diseases such as cancer. In some embodiments, FETS comprising both n-type and p-type Si nanowires with different receptors within the same sensor may be required for reliable cancer and other disease detection.

In some instances, the high ionic strength environment in a health and wellness analytical toilet from excreta may adversely affect the accuracy and precision of the FET-based biomarker sensor. In some embodiments, a biomolecule permeable layer may be located over the sensor. The biomarker permeable layer may be substantially impermeable to ions such that only biomarkers are able to pass through the layer and approach the sensor. The layer may increase the effective Debye screening length in the region immediately adjacent to the sensor surface. This may allow detection of biomolecules in high ionic strength solutions in real-time. In some embodiments, the layer may only be permeable to a target analyte. In some embodiments, the layer may be only permeable to a class of analytes. The layer may be comprised of a membrane. The layer may be porous. The layer may be comprised of a polymer. The polymer may be comprised of polyethylene glycol.

### FET-Based Sensor Fluidic System

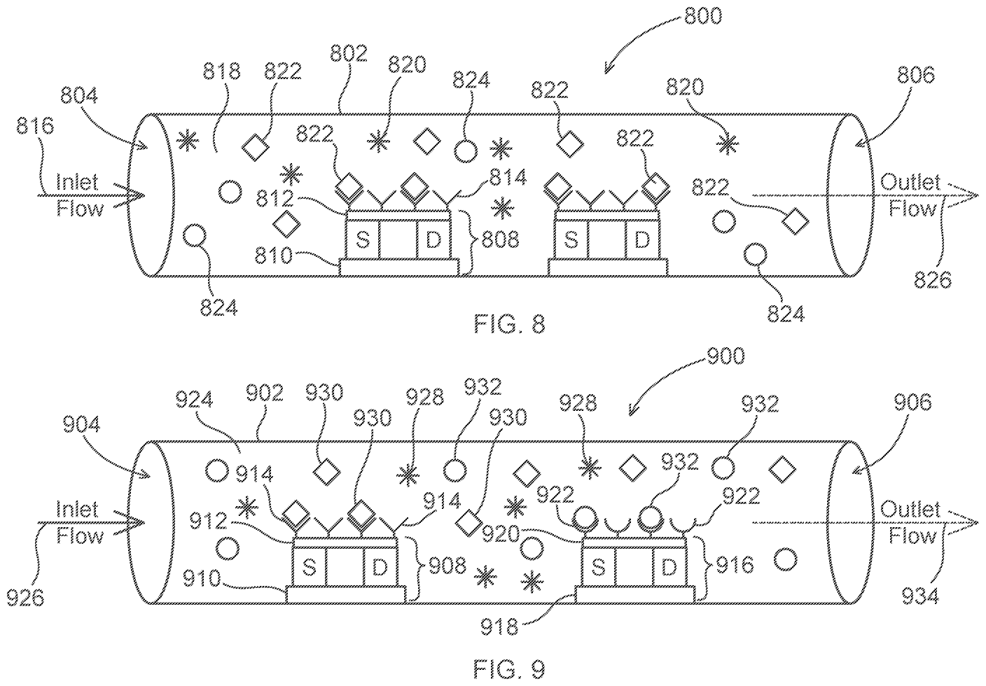

FIG. 8 illustrates sample flow to a FET-based sensor in a fluidic system, according to an embodiment of the disclosure. Fluidic system is designed to detect a single analyte. Fluidic system 800 in FIG. 8 comprises a passageway 802 by which a sample may pass through. Passageway 802 may be a fluidic or microfluidic channel. Passageway 802 comprises an inlet 804 and outlet 806. Within passageway 802 is one or more FET-based sensors 808. FET-based sensor 808 comprises a source (S) terminal and drain (D) terminal on a support, body 810. Sensor 808 further comprises a gate component 812. Gate component 812 bridges the S and D terminals. Gate component 812 may comprise a nanowire, nanocrystal, conducting polymer, p-Si or n-Si or other component as previously described herein. Although only two FET sensors are shown in fluidic system embodiment 800, two or more FET sensors may be located in the system. The FETs may be organized in an array-like manner. Fluidic system 800 may comprise multiple channels with each channel comprising one or more FET sensors. A single feed channel from the toilet may be combined with a plenum to feed a sample into two or more channels wherein each channel composes one or more FET-based sensors.

Gate component 812 is functionalized with one or more receptors 814. Receptor 814 may be an antibody, monoclonal antibody, PNA, nucleic acid, RNA, DNA or other type of receptor as previously described herein. In an exemplary embodiment, receptor 814 is specific to an analyte or a portion of an analyte, such as a biomarker.

A sample flow may pass through passageway 802 in a continuous or segmented flow. Inlet flow 816 of a sample of excreta 818 is represented by a solid line arrow. Excreta 818 typically contains a variety of analytes such as ions 820, biomarker molecules 822 and non-biomarker molecules 824. As the flow of excreta 818 passes through passageway 802, the analytes interact with the receptors 814 located on gate components 812 in the FETs 808. When a target analyte interacts with a specially designed receptor 814, the electronic properties of the gate component in the FET are altered in a measurable fashion. In fluidic system 800 shown in FIG. 8, target biomarker analyte 814 (diamond-shaped) binds with a receptor 814. The other non-target analytes 820, 824 do not interact with the receptors 814. The excreta sample 818 continues to flow through passageway 802 and exits the passageway at outlet 806 as outlet flow 826 (dotted line). In some embodiments, outlet, flow 826 of sample 818 may be directed to other passageways and sensors that may detect other analytes.

FIG. 9 illustrates sample flow to a FET-based sensor in another fluidic system, according to an embodiment of the disclosure. Fluidic system 900 is designed to detect a first analyte and a second different analyte. Fluidic system 900 in FIG. 9 comprises a passageway 902 by which a sample may pass through. Passageway 902 may be a fluidic or microfluidic channel. Passageway 902 comprises an inlet 904 and outlet 906.

Within passageway 902 is a first FET-based sensor 908 that is designed to detect a first analyte. FET-based sensor 908 comprises a source (S) terminal and drain (D) terminal on a support body 910 Sensor 908 further comprises a gate component 912. Gate component 912 bridges the S and D terminals. Gate component 912 may comprise a nanowire, nanocrystal, conducting polymer, p-Si or n-Si as previously described herein. Although only two FET sensors are shown in fluidic system embodiment 900, two or more FET sensors may be located in the system. The FETs may be organised in an array-like manner. Fluidic system 900 may comprise two or more channels with each channel comprising one or more FET sensors. A single feed channel from the toilet may be combined with a plenum to feed a sample into two or more channels wherein each channel comprises one or more FET-based sensors.

Gate component 912 is functionalized with one or more receptors 914. Receptor 914 may be an antibody, monoclonal antibody, PNA, nucleic acid, RNA, DNA or other type of receptor as previously described herein. In an exemplary embodiment, receptor 914 is specific to an analyte or a portion of an analyte, such as a biomarker.

Within passageway 902 is a second FET-based sensor 916 that is designed to detect a second and different analyte. FET-based sensor 916 comprises a source (S) terminal and drain (D) terminal on a support body 918. Sensor 916 further comprises a gate component 920. Gate component 920 bridges the S and D terminals. Gate component 920 may comprise a nanowire, nanocrystal, conducting polymer, p-Si or n-Si as previously described herein.

Gate component 920 is functionalized with one or more receptors 922. Receptor 922 may be an antibody, monoclonal antibody, PNA, nucleic acid, RNA, DNA or other type of receptor as previously described herein. In an exemplary embodiment, receptor 922 is specific to an analyte or a portion of an analyte, such as a biomarker.

A sample flow may pass through passageway 902 in a continuous or segmented flow. Inlet flow 926 of a sample of excreta 924 is represented by a solid line arrow. Excreta 924 typically contains a variety of analytes such as ions 928, biomarker molecules 930 and non-biomarker molecules 932. As the flow of excreta 924 passes through passageway 902, the analytes interact with the receptors 914 located on gate components 912 in the first FET 908. When a target analyte interacts with a specially designed receptor 914, the electronic properties of the gate component in the first FET 908 are altered in a measurable fashion. In fluidic system 900 shown in FIG. 9, target biomarker analyte 930 (diamond-shaped) binds with a receptor 914. The other non-target analytes 928, 932 do not interact with the receptors 914. The excreta sample 924 continues to flow through passageway 902 towards a second FET-based sensor 916. Target analyte 932 interacts and binds with receptor 922. The other analytes 928, 930 do not bind with receptor 922 and pass on by. In this fluidic system, two specific analytes are detected. In other embodiments, a fluidic system may be designed to detect three or more different analytes.

The flow of sample of excreta 924 exits the passageway at outlet 906 as outlet flow 934. In some embodiments, outlet flow 934 of sample 924 may be directed to other passageways and sensors that may detect other analytes.

### Sample Delivery Methods to the Sensor

Once excreta has been deposited in the toilet, there are many ways the excreta could be processed in preparation for testing by a FET-based sensor system, such as the fluidic system embodiments illustrated in FIGS. 8-9. Some pretreatments include a filter, a centrifuge, dilution, or pH normalization. In one preferred embodiment, a portion of feces is separated from urine, mixed with water and/or a reagent, and presented to the component of a sensor for analysis. Following analysis, the sample is removed from the sensor, and the sensor is cleaned and/or sterilized in preparation for a new sample being presented to the component of the sensor.

Once the sample is prepared, several methods may be used to deliver the sample to the sensor. A first method comprises delivering a continuous inlet stream of sample to the sensor. The continuous stream may be delivered over a pre-determined length of time. One or more tests may be carried out during the length of time the sample is passed over the sensing component (i.e., nanowire, nanocrystal, conducting polymer, etc.) of the FET-based sensor. The length of time for continuous flow may be about 0.1 sec or greater. In other embodiments, the time may be in the range of about 0.1-10 sec. In other embodiments the time may be in the range of about 0.1-5 sec. In other embodiments, the time may be about 0.1-1 sec. The length of time may be sample dependent such that the length of time depends on the volume of sample of excreta wherein the continuous flow is carried out until the sample is depleted. The length of time may also depend on the required residence time the target analyte needs to interact and bind with a receptor in the sensor. This may be analyte/sensor dependent. The rate of sample flow to one or more sensors may be greater than about 1 nL/sec. The rate of sample flow may be in the range of about 1 μL/sec to about 10 mL/sec. The rate of sample flow may be in the range of about 1 μL/sec to about 1 mL/sec. The rate of sample flow may be in the range of about 1-100 μL/sec. The rate of sample flow may be in the range of about 1-10 μL/sec.

A second method to deliver the sample of excreta is a segmented flow wherein the sample is delivered in portions to a sensor. The portions may be pre-determined measured volumes. This may aid in understanding the concentrations of analytes that are present in the sample. Each portion delivered to a sensor may then be allowed to have a residence time for the sensor to interact with a target analyte. The analyte/receptor interaction kinetics may have to be determined to choose a specific residence time.

Each sample portion may be followed by a portion of a cleaning solution In an alternating fashion in order to clean the sensor before each test. A cleaning solution may be needed to remove bound analytes to the receptors of the sensors. A cleaning solution may be delivered to the sensor after a pre-determined number of sample portions have been delivered to the sensor. For example, a cleaning solution may be delivered to the sensor after every two, three, four, five or more samples of excreta that have been delivered to and tested by the sensor. The cleaning solution may be deionized water, a buffer solution or a solution comprising one or more known control analytes. The cleaning solution may comprise an organic material such as methanol, ethanol or other hydrocarbons. The sample of excreta portion may be alternated with a portion of air. The portions may be delivered to the sensor using a pump, such as a peristaltic pump. The number of sample portions may be or may not be equal to the number of analyte detection tests desired. The volume of the sample and cleaning portions may be approximately the same or not the same. The volume of the sample or cleaning portions may be greater than about 1 nL. In some embodiments, the volume of the sample and cleaning portions may be in the range of about 1 nL to about. 200 ml. The volume of the sample and cleaning portions may be in the range of about 1 nL to about 1 ml. The volume of the sample and cleaning portions may be in the range of about 1 nL to about 1 μl. The rate of delivery of a portion of a sample in a segmented flow method to one or more sensors may be greater than about 1 nL/sec. The rate of delivery of a single portion may be in the range of about 1 μL/sec to about 10 mL/sec. The rate of a delivery of a portion may be in the range of about 1 μL/sec to about 1 mL/sec. The rate of delivery of a portion of sample may be in the range of about 1-100 μL/sec. The rate of delivery of a portion of sample may be in the range of about 1-10 μL/sec.

In some embodiments, a calibration standard may be delivered to the sensor after each test. This helps to assure that the sensor is functioning properly before each test. In some embodiments, a calibration standard may be used after every two, three, four, five or more tests. This is dependent upon how stable the sensor device is in the sample environment. In some embodiments, a cleaning solution may first be delivered to the sensors to substantially remove any bound analytes followed by a solution comprising a calibration standard. In some instances, cleaning and calibration steps may need to be repeated two or more times to assure that the sensors are cleaned of analytes before the next user utilized the analytical toilet. The number of cleaning/calibration steps may need to be repeated until the calibration data is within a pre-determined value.

Following use of the sensor, the toilet may prepare the sensor for future analysis by removing from the test area waste products and other things that might contaminate the next analysis. This could include flushing the sensor, adding a buffer or stabilizing solution, or adding a gas to remove all liquid from the sensor. There are various options to clean, sanitize, and/or prepare the various components of the involved between uses of the toilet. In one preferred embodiment, hot water is run through the fluidic circuit. In another preferred embodiment, oxygenated water is run through the fluidic circuit. In yet another preferred embodiment, a gas is run through the fluidic circuit to remove any liquid from being in contact with the sensor. Alternatively, cleaning and/or preservation agents are run through the fluid circuit. In still another embodiment, if an analyte receptor, such as an antibody receptor, is used in one or more sensors, the sensors are washed with a solution comprising one or more molecules at a predetermined concentration that can interact with and bind with the receptors in a known and predictive manner. This may be necessary when water or other solvent alone may not be sufficient to displace bound analytes, such as biomarkers, in order to clean the sensor. This cleaning method can act as an indicator to show that the sensors are washed and cleared of analytes before the next subject utilizes the toilet. The analytes may be further cleared from the sensor components using a cleaning or preservation agent dispensed from the toilet.

Additionally, temperature can be critical to the preparation, testing, or post processing of the sensor, the fluidic circuit, or the sample. As such, temperature controls may be included to accommodate those need. The controls could be built into the toilet, built into a fluidic circuit, or a result of adding a reagent to the sample. In one preferred embodiment, a resistive wire acts as a heat source to warm the sample and/or the sensor.

### Analysis of FET-Based Sensor Data

A processor analyzes sensor data once the processor begins to receive the data. If any data is outside the range of the detection limits of the sensor, the sample may further be diluted or concentrated depending on the data. For example, if the sensor data appears to be too high, such as a conductance level is detected above a maximum and reliable limit, the sample may be diluted. Dilution the sample may lower the conductance value within a reliable range. Dilution of the sample may be carried out with a buffer solution, deionized water or other suitable diluent. In some cases, the sensors may detect a target analyte that is below a minimum and reliable limit. In this instance, a heater may be used to drive off a portion of the fluid carrier, such as water from the sample, until the sensor data is within a reliable and pre-determined range. A desiccant may also be used to remove a portion of the fluid carrier of the sample.

Once the testing parameters are reached and reliable data is collected that is within a pre-determined range, the data may then be statistically evaluated. The mean, median, and standard deviation of the data may be carried out. Additionally, regression analysis may be carried out on the data of a single user. Regression analysis may also be used on two or more users to understand how the data of a single user compares to a population of users of the analytical toilet.

A Q test may be carried out on the data of a user. A Q test is used to determine if any of the data comprises an outlier. An outlier may result due to an error in the operation of the toilet, such as due to a mechanical breakdown or the incorrect use of a cleaning or calibration solution or other reason. If an outlier is detected, the test may be repeated. Each time the test is repeated, statistical analysis on the data of the user may also be repeated. The test may be repeated until the minimum number of tests is attained and wherein the data is within a reliable range of high confidence.

In some embodiments, FET-based sensors in an analytical toilet described herein may further be combined with other methods of biomarker detection. Additional biomarkers may be measured via a miniaturized mass spectrometer. Alternatively, additional biomarkers may be measured using gas chromatography integrated into the toilet body or positioned adjacent to the toilet. Additional biomarkers may also be measured using fluorescence spectrometry. A fluorescent tag may be covalently or ionically attached to a target molecule. These tags may be a protein, antibody, peptide or amino acid. These tagged molecules may then be used to detect a specific target such as an antigen. In some instances, two or more detection methods, such as those described herein, may be used to detect the same biomarker.

In various exemplary embodiments, an analytical toilet comprising FET-based sensors may be located in a variety of location. In some embodiments, the seat may contain health and wellness sensors to measure pulse, blood pressure, blood oxygenation, electrocardiography, body temperature, body weight, excreta content, excreta weight, excreta volume, excreta temperature, excreta density, excreta flow rate, and other health and wellness indicators. In a preferred embodiment, the seat is attached to the toilet via a powered quick disconnect system that allows the seat to be interchangeable. This facilitates installing custom seats to include user-specific tests based on known health conditions. It also facilitates installing upgraded seats as sensor technology improves.

In various exemplary embodiments, the lid may contain health and wellness sensors that interact with the user's back or that analyze gases in the bowl after the lid is closed.

In various exemplary embodiments, the analytical toilet includes software and hardware controls that are pre-set so that any manufacturer can configure their devices (i.e., analytical test devices) to work in the system. In a preferred embodiment, the system includes a software stack that allows for data channels to transfer data from the sensors in the medical toilet to cloud data systems. The software and hardware controls and/or software stack may be stored in the analytical toilet or remotely. This would allow scientists to place sensors, reagents, etc. in the system to obtain data for their research. It also allows user data to be individually processed, analyzed, and delivered to the user, or their health care provider, digitally (e.g., on a phone, tablet, or computer application). The seat may also contain sensors to measure fluid levels in the toilet. This could include proximity sensors. Alternatively, tubes in fluid communication with the bowl water could be used to determine changes to bowl fluids (e.g., volume, temperature, rate of changes, etc.).

The toilet disclosed herein has many possible uses, including private and public use. Whether for use by one individual, a small group of known users, or general public use, the toilet can detect, monitor, and create one-time and/or trend data for a variety of analytes, such as biomarkers. This data can be used to prompt a user to seek additional medical, health, or wellness advice or treatment; track or monitor a user or population's known condition; and provide early detection or anticipation of a disease or another condition of which a user or population may wish to be aware.

While the present disclosure often notes the sensor and other equipment supporting excreta analysis are located within the toilet, it is possible that some or all of the components are located outside of the toilet. For example, the sample preparation, detection, and processing equipment may be a separate unit adjacent to the toilet which cooperates with the toilet to automatically or semi-automatically receive excreta, prepare a sample of excreta for analysis, test the sample, discard the sample, and prevent cross contamination by cleaning and/or sterilizing portions of the toilet and external equipment that do any portion of the described process.

### EXAMPLE

The following example is provided as part of the disclosure as an embodiment of the present invention. As such, none of the information provided below is to be taken as limiting the scope of the invention.

### Example 1

### Detecting a SARS-COV-2 (COVID-19) Virus

Example 1 is illustrative of a preferred method of detecting a virus. The method comprises:
1) A user releases a sample of excreta into an analytical toilet.
2) A microfluidic system within the analytical toilet directs and transports a sample of the excreta to a sensor. A component of the sensor comprises a FET with a silicon nanowire of approximately 10 nm in diameter that bridges the source and drain electrodes. The nano wire is functionalized with the SARS-COV-2 specific antibody ACE2 (ProSci, Inc., Poway, Calif., USA).
3) A 10 μL sample of excreta is delivered over a period of 1 sec to the FET sensor. The sample is held in place for a residence time of 60 sec.
4) The sensor detects a change in conductance in the FET due to an interaction event of SARS-COV-2 (COVID-19) virus with the ACE2 antibody bound to the nanowire.
5) The sensor relays the computer-readable data to a processor.
6) The processor processes the data and stores the data.
7) A 10 mL cleaning solution sample is passed over the sensors to remove the sample from the sensors.
8) A 10 μL solution of calibration standard is delivered over a period of 1 sec to the FET sensor. The standard is held in place for a residence time of 10 sec. The sensor detects a change in conductance in the FET due to an interaction event with the calibration standard. The processor processes the data and stores the data. The calibration data is within a pre-determined value which indicates that the sensors are cleaned and operating correctly.
9) A second 10 μL sample of excreta is delivered over a period of 1 sec to the FET sensor. The sample is held in place for a residence time of 60 sec.
10) The sensor detects a change in conductance in the FET due to an interaction event of SARS-COV-2 (COVID-19) virus with the ACE2 antibody bound to the nanowire.
11) The sensor relays the computer-readable data to a processor.
12) The processor processes the data and stores the data.
13) A second 10 mL cleaning solution sample is passed over the sensors to remove the sample from the sensors.
14) A second 10 μL solution of calibration standard is delivered over a period of 1 sec to the FET sensor. The standard is held in place for a residence time of 10 sec. The sensor detects a change in conductance in the FET due to an interaction event with the calibration standard. The processor processes the data and stores the data. The calibration data is within a pre-determined value which indicates that the sensors are cleaned and operating correctly.
15) A third 10 μL sample of excreta is delivered over a period of 1 sec to the FET sensor. The sample is held in place for a residence time of 60 sec.
16) The sensor detects a change in conductance in the FET due to an interaction event of SARS-COV-2 (COVID-19) virus with the ACE2 antibody bound to the nanowire.
17) The sensor relays the computer-readable data to a processor.
18) The processor processes the data and stores the data.
19) A third 10 mL cleaning solution sample is passed over the sensors to remove the sample from the sensors.
20) A third 10 μL solution of calibration standard is delivered over a period of 1 sec to the FET sensor. The standard is held in place for a residence time of 10 sec. The sensor detects a change in conductance in the FET due to an interaction event with the calibration standard. The processor processes the data and stores the data. The calibration data is within a pre-determined value which indicates that the sensors are cleaned and operating correctly.
21) The data from the tests is processed by a processor. The mean, median, standard deviation, regression and outlier tests are carried out on the sensor data. No outlier data is detected. The sensor tests are not repeated.
22) The data is delivered to the user or medical professional and takes appropriate action in response to the data.
23) The analytical toilet flushes and cleans the bowl in preparation for the next user.
24) The sensors are cleaned with a cleaning solution followed by a solution containing a calibration standard until the calibration data is within a pre-determined value. Once the pre-determined value is reached, the calibration standard solution is replaced with a buffer solution engineered to preserve and protect the functionalized nanowire sensor until the next user uses the analytical toilet.

All patents, published patent applications, and other publications referred to herein are incorporated herein by reference. The invention has been described with reference to various specific and preferred embodiments and techniques. Nevertheless, it is understood that many variations and modifications may be made while remaining within the spirit and scope of the invention.

What is claimed is:

1. A method to detect an analyte in an analytical toilet comprising:
receiving excreta in a bowl;
transporting a measured sample of the excreta through a passage; and
bringing the sample into contact with a sensor;
wherein the sensor comprises a FET configured to interact with an analyte in the excreta; and
wherein, when the sample is brought into contact with the sensor, the sensor indicates the presence of the analyte by a distinct electric signal.

2. The method of claim 1 wherein a property of the electric signal is indicative of the concentration of the analyte in the sample.

3. The method of claim 1 wherein the FET comprises a nanowire functionalized to interact with the analyte.

4. The method of claim 3 wherein the analyte is selected from the group consisting of viruses, bacteria, DNA, RNA, proteins, nucleic acids, amino acids, peptides, polysaccharides, ions, and fragments thereof.

5. The method of claim 1 wherein the analyte is selected from the group consisting of viruses, bacteria, DNA, RNA, proteins, nucleic acids, amino acids, peptides, polysaccharides, ions, pharmaceutical compounds, and fragments and metabolites thereof.

6. The method of claim 1 wherein the sensor is located on a sensor element, wherein the toilet comprises a slot for receiving the sensor element, and wherein the slot provides an interface whereby, when the sensor element is inserted in the slot, the sensor is aligned with the passage and provided with electrical power and data communication.

7. The method of claim 6 wherein the sensor element comprises microfluidic channels to transport the sample to the sensor.

8. The method of claim 7 wherein the sensor element further comprises additional microfluidic channels to transport a cleaning fluid to the sensor.

9. The method of claim 1 wherein the transport of the sample of excreta to the sensor is by continuous flow or segmented flow.

10. The method of claim 9 wherein the segmented flow comprises a segment of a sample of excreta and a segment of buffer.

11. The method of claim 10 wherein the segments are measured portions.

12. The method of claim 1 wherein the sensor is cleaned after each time the sensor is exposed to a sample of excreta.

13. The method of claim 1 wherein the sensor is calibrated after each time the sensor is exposed to a sample of excreta.

14. The method of claim 1 wherein when two or more samples of excreta are tested, the resulting data undergoes statistical analysis by a processor to compile statistical data.

15. The method of claim Error! Reference source not found, wherein the statistical data is used to generate individual trend data over time of the analyte in the excreta of a particular user.

16. The method of claim 15 wherein the individual trend data is reported to the user to enhance health and wellness.

17. The method of claim 15 wherein data from the sensor is used to monitor for or track a specific health and wellness condition.

18. The method of claim 1 wherein the sensor can interact with two or more different analytes at the same time.

19. The method of claim 1 wherein the sensor further comprises a layer that is permeable to a biomolecule.

20. The method of claim 1 wherein if the electrical signal is not within a pre-determined range of the sensor, the sample of excreta is adjusted and re-tested until the electrical signal is within the pre-determined range of the sensor.
